(19) United States
(12) Patent Application Publication
(10) Pub. No.: US 2014/0113374 A1
(43) Pub. Date: Apr. 24, 2014

(54) CELL LINES THAT OVEREXPRESS LACTATE DEHYDROGENASE C

(71) Applicant: REGENTS OF THE UNIVERSITY OF MINNESOTA, Saint Paul, MN (US)

(72) Inventors: Wei-Shou Hu, Falcon Heights, MN (US); Bhanu Chandra Muhukutla, Lawrence, MA (US); Anne Kantardjieff, Milford, CT (US)

(21) Appl. No.: 13/990,716

(22) PCT Filed: Nov. 30, 2011
(86) PCT No.: PCT/US11/62615

§ 371 Date: Aug. 19, 2013

Related U.S. Application Data
(60) Provisional application No. 61/418,071, filed on Nov. 30, 2010.

(51) Int. Cl. C12N 9/04

(52) U.S. Cl. CPC C12N 9/0006
USPC 435/455; 435/320.1; 435/252.33; 435/471; 435/326; 435/358

(57) ABSTRACT

Cell lines that overexpress lactate dehydrogenase C (LDH-C) are disclosed. Generally, the cells include a heterologous polynucleotide molecule that encodes a LDH-C polypeptide or a fragment or biologically active analog that possesses measurable LDH-C activity. Also disclosed is a method of increasing the productive yield of a cell that produces a therapeutic agent. Generally, the method includes introducing into the cell a heterologous polynucleotide molecule that encodes a LDH-C polypeptide or a fragment or biologically active analog that possesses measurable LDH-C activity.

### CROSS-REFERENCE TO RELATED APPLICATION

This application claims priority to U.S. Provisional Patent Application Ser. No. 61/418,071, filed Nov. 30, 2010.

### BACKGROUND

Mammalian cells in culture display extensive glycolysis and convert most of the glucose they consume to lactate. This inefficient nutrient utilization can lead to lactate accumulation, which can be detrimental to cell growth and result in decreased maximum achievable cell density

### SUMMARY OF THE INVENTION

In one aspect, the invention provides an isolated polynucleotide molecule. Generally, the isolated polynucleotide molecule includes a coding region that encodes at least a portion of a lactate dehydrogenase C polypeptide, operably linked to a promoter.

In some embodiments, the coding region can include at least a portion of SEQ ID NO:4, SEQ ID NO:6, SEQ ID NO:10, SEQ ID NO:13, SEQ ID NO:15, SEQ ID NO:17, SEQ ID NO:19, or SEQ ID NO:21 that encodes at least a portion of a lactate dehydrogenase C polypeptide.

In some embodiments, the coding region encodes the amino acid sequence of SEQ ID NO:5, SEQ ID NO:7, SEQ ID NO:8, SEQ ID NO:9, SEQ ID NO:11, SEQ ID NO:14, SEQ ID NO:16, SEQ ID NO:18, SEQ ID NO:20, or SEQ ID NO:22.

In another aspect, the invention provides a genetically modified cell. In some embodiments, the genetically modified cell exhibits greater lactate dehydrogenase C activity compared to a wild-type control. In other embodiments, the genetically modified cell consumes lactate to a greater degree than a wild-type control. In some embodiments, the genetically modified cell produces lactate to a lesser degree than a wild-type control. In other embodiments, the genetically modified cell exhibits a greater maximum cell density when grown in nutrient-rich medium compared to a wild-type control. In various embodiments, the genetically modified cell can exhibit any combination of two or more of the foregoing characteristics.

In some embodiments, the genetically modified cell includes a cell and a heterologous polynucleotide molecule that comprises a coding region that encodes at least a portion of a heterologous lactate dehydrogenase C polypeptide. In other embodiments, the genetically modified cell includes a lactate dehydrogenase C coding region and an endonuclease modification that results in the lactate dehydrogenase C coding region being expressed to a greater degree than a comparable cell without the endonuclease modification.

In some embodiments, the genetically modified cell expresses the heterologous lactate dehydrogenase C polypeptide to a greater extent than the cell, without the genetic modification, expresses lactate dehydrogenase C. In other embodiments, the genetically modified cell consumes lactate to a greater degree than the cell, without the genetic modification, consumes lactate. In other embodiments, the genetically modified cell produces lactate to a lesser degree than the cell, without the genetic modification, produces lactate. In still other embodiments, the genetically modified cell possesses a greater maximum cell density than the maximum cell density of the cell, without the genetic modification.

In some embodiments, the genetically modified cell can further include an inhibiting polynucleotide molecule that inhibits expression of lactate dehydrogenase A.

In some embodiments, the genetically modified cell can include a coding region that includes at least a portion of SEQ ID NO:4, SEQ ID NO:6, SEQ ID NO:10, SEQ ID NO:13, SEQ ID NO:15, SEQ ID NO:17, SEQ ID NO:19, or SEQ ID NO:21 that encodes at least a portion of a lactate dehydrogenase C polypeptide. In other embodiments, the genetically modified cell can include a coding region that encodes the amino acid sequence of SEQ ID NO:5, SEQ ID NO:7, SEQ ID NO:8, SEQ ID NO:9, SEQ ID NO:11, SEQ ID NO:14, SEQ ID NO:16, SEQ ID NO:18, SEQ ID NO:20, or SEQ ID NO:22.

In some embodiments, the genetically modified cell can further exhibit reduced lactate dehydrogenase A activity compared to a wild-type control. In some of these embodiments, the genetically modified cell can further include a heterologous polynucleotide sequence that encodes a therapeutic agent.

In another aspect, the invention provides a method that generally includes providing a cell that produces a therapeutic product; and introducing into the cell an isolated polynucleotide, thereby producing a transformed cell. Generally, the isolated polynucleotide molecule includes a coding region that encodes at least a portion of a lactate dehydrogenase C polypeptide, operably linked to a promoter. In some embodiments, the coding region can include at least a portion of SEQ ID NO:4, SEQ ID NO:6, SEQ ID NO:10, SEQ ID NO:13, SEQ ID NO:15, SEQ ID NO:17, SEQ ID NO:19, or SEQ ID NO:21 that encodes at least a portion of a lactate dehydrogenase C polypeptide. In some embodiments, the coding region encodes the amino acid sequence of SEQ ID NO:5, SEQ ID NO:7, SEQ ID NO:8, SEQ ID NO:9, SEQ ID NO:11, SEQ ID NO:14, SEQ ID NO:16, SEQ ID NO:18, SEQ ID NO:20, or SEQ ID NO:22.

In some embodiments, introducing the isolated polynucleotide into the cell causes the cell to increase expression of lactate dehydrogenase C. In other embodiments, introducing the isolated polynucleotide into the cell causes the cell to decrease production of lactate. In other embodiments, introducing the isolated polynucleotide into the cell causes the cell to increase consumption of lactate. In still other embodiments, introducing the isolated polynucleotide into the cell causes an increase in the cell's maximum cell density.

In yet another aspect, the invention provides a method that generally includes growing a cell in culture comprising culture medium comprising glucose, and controlling glucose concentration in the culture medium so that the culture exhibits at least one of the following characteristics compared to growth of the cell in culture medium comprising at least 2.0 g/L glucose: reduced lactate accumulation, enhanced lactate consumption, increase cell density, or increased viability.

In some embodiments, initiating control of the glucose concentration can begin at a time when or after the growth rate of the culture falls from a maximum growth rate to 20% of the maximum growth rate.

In some embodiments, the cell culture can include a genetically modified cell according to any embodiment summarized above.

In some embodiments, control of the glucose concentration can include maintaining the glucose concentration of the culture medium at no more than 0.5 g/L.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Growth characteristics and lactate profiles of LDH-C overexpression clone (▴) and parental cell line (♦).

FIG. 2. Growth characteristics and lactate profiles of clones with LDH-C overexpression over LDH-A knockdown background (▪), clone with LDH-A knock down (♦), and parental cell line (▴).

FIG. 3. Bioreactor data for low glucose-mediated metabolic shift to lactate consumption (♦) in Chinese hamster ovary (CHO) cell line compared to high glucose (▪) culture. (A) cell density; (B) duration of cell viability, (C) lactate concentration, (D) glucose concentration, (E) amount of model recombinant protein produced. The initiation of glucose control is indicated in panel (A).

FIG. 4. Bioreactor data for low glucose mediated metabolic shift to lactate consumption in mouse myeloma (SP2/0) cell line.

### DETAILED DESCRIPTION OF ILLUSTRATIVE EMBODIMENTS

Mammalian cells in culture display enhanced glycolysis and convert most of the glucose they consume to lactate. This inefficient use of nutrients can lead to lactate accumulation, which has been shown to be detrimental to cell growth and reduced maximum cell densities.

The present invention relates to cell lines, isolated polynucleotides, and methods related to overcoming the accumulation of lactate in cell cultures. In general terms, the present invention relates to tools for modifying the metabolism of cultured cells so that the cells consume lactate, the culture exhibits reduced lactate accumulation in the culture medium, and/or the culture possesses a greater maximum cell density. In embodiments in which the cell culture is further designed to produce a therapeutically active agent such as, for example, a therapeutic protein, vaccine component, and the like, the invention can increase production of the agent by, for example, increasing the density of agent-producing cells in the culture and/or maintaining a late-stage culture environment conducive to producing the agent.

As used herein, the following terms shall have the indicated meanings.

“Coding region” refers to the portion of a polynucleotide sequence that encodes a structural polypeptide amino acid sequence as opposed to, for example, 3′ non-coding regions such as, for example, ribosome binding sites and 5′ noncoding regions such as, for example, polyadenylation.

“Heterologous” refers to a polynucleotide molecule or polypeptide originating from outside a cell of reference. Thus, as used herein, a heterologous polynucleotide molecule or polypeptide can include a polynucleotide sequence or polypeptide originating from an organism of a different species or another organism of the same species. As used herein, a heterologous polynucleotide molecule also can include a polynucleotide molecule synthesized outside of the cell of reference, and may include, for example, one or more additional copies of a polynucleotide sequence synthesized in vitro and/or in another cell of the organism from which the reference cell is derived. A heterologous polypeptide can include a polypeptide synthesized by the cell of reference by standard protein expression from a heterologous polynucleotide molecule.

“Inhibiting polynucleotide molecule” refers to a polynucleotide molecule that, when introduced into a cell, can inhibit translation of an mRNA targeted by the inhibiting polynucleotide molecule. The inhibiting polynucleotide molecule may be RNA such as, for example an siRNA, miRNA, shRNA, or antisense RNA; a DNA that either inhibits translation of a target mRNA or encodes an RNA that inhibits translation of a target mRNA; or a DNA that encodes a polypeptide that inhibits translation of a target mRNA.

“Lactate dehydrogenase C polypeptide” refers to any polypeptide identified as lactate dehydrogenase C, regardless of the species of origin, including any fragment or analog thereof that possesses measurable lactate dehydrogenase C activity.

“Therapeutic agent” refers to a cellular product having therapeutic activity. A therapeutic agent may be, for example, a recombinant protein or more complex such as, for example, a viral vaccine.

The term “and/or” means one or all of the listed elements or a combination of any two or more of the listed elements.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

Initially, we compared the expression profiles of cultured cells and their tissue of origin. We found that cultured cells have upregulated a number of glycolytic enzymes, including lactate dehydrogenase A (LDH-A).

Consequently, we used short interfering RNAs (siRNAs) to downregulate LDH-A in an antibody-producing mouse myeloma SP2/0 cell line, and achieved more than 80% reduction at the enzyme activity level in one of the characterized clones.

We also examined the effects of overexpressing the LDH-C isoform, whose kinetic properties favor conversion of lactate to pyruvate, and which could potentially result in a lactate consuming phenotype. LDH-C overexpression alone, as well as LDH-C overexpression combined with an LDH-A knockdown, yielded appreciable changes in central metabolism, manifested as reduced lactate accumulation, lactate consumption, and/or increased maximum cell densities.

Initial studies revealed upregulation of the glycolytic pathway of cultured cell lines compared to their tissue of origin. Upregulation was visualized using GenMAPP (University of California at San Francisco, San Francisco, Calif.), which allows for microarray data to be overlaid onto canonical metabolic and signaling pathways. Examination of the lactate dehydrogenase LDH node reveals that the LDH-A isoform is upregulated in both SP2/0 and Chinese hamster ovary (CHO) cell lines compared to their respective tissue sources, mouse plasma cells and Chinese hamster ovary tissue, respectively. In SP2/0 cells, the LDH-A subunit is predominantly expressed, with an average intensity of 5200, compared to an average intensity of 80 for the LDH-B subunit. The LDH-C subunit is not natively expressed in SP2/0 cells. In CHO cells, LDH-A is expressed at much higher levels (˜50-fold higher) as compared to lower expression of LDH-C. LDH-B is not expressed in CHO cells.

The lactate dehydrogenase enzyme catalyzes the reversible conversion of pyruvate to lactate, accompanied by the oxidation of NADH to NAD+. The enzyme exists as a tetramer with varying composition of two subunits: LDH-A and LDH-B. A third isoform, composed exclusively of subunits encoded by the LDH-C gene, is found exclusively in the testis.

Each LDH isoform has distinct kinetic properties. Consequently, there is tissue-specific distribution of each LDH isoform, according to the metabolic requirements of each tissue. For instance, the LDH-1 isoform, composed exclusively of LDH-B subunits, is the predominant subunit in mainly aerobic tissues, such as cardiac muscle. In the testes, the LDH-C isoform is predominant and lactate in involved in the energy metabolism of Sertoli and germ cells. Sertoli cells, which predominantly express the LDH-5 isoform, convert pyruvate to lactate, which is subsequently transported into germ cells via monocarboxylate transporters. Lactate is then converted to pyruvate by the LDH-C enzyme, and pyruvate enters the TCA cycle for energy generation.

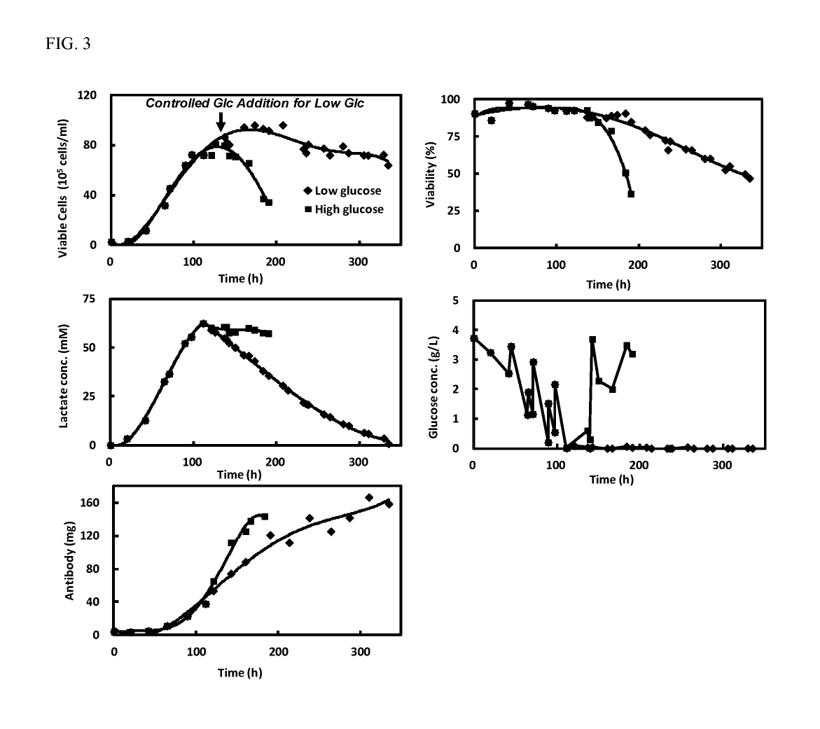

Based on the observations of our transcriptome comparison of cultured mammalian cells to their native tissues, we engineered cellular metabolism by modulating expression levels of the various LDH isoforms. We engineered cell lines exhibiting knocked down LDH-A and LDH-B transcript levels in cultured mammalian cells with the aim of reducing lactate formation and ameliorating nutrient utilization for increased energy production. To accomplish this, we used siRNA (short interfering RNA) technology. (Example 1, Example 3, and FIG. 2). By generating stable transfectants, we were able to isolate clones with more than eight-fold downregulation of the LDH-A transcript, and greater than 80% reduction at the enzyme activity level. These were characterized in terms of their growth and nutrient consumption.

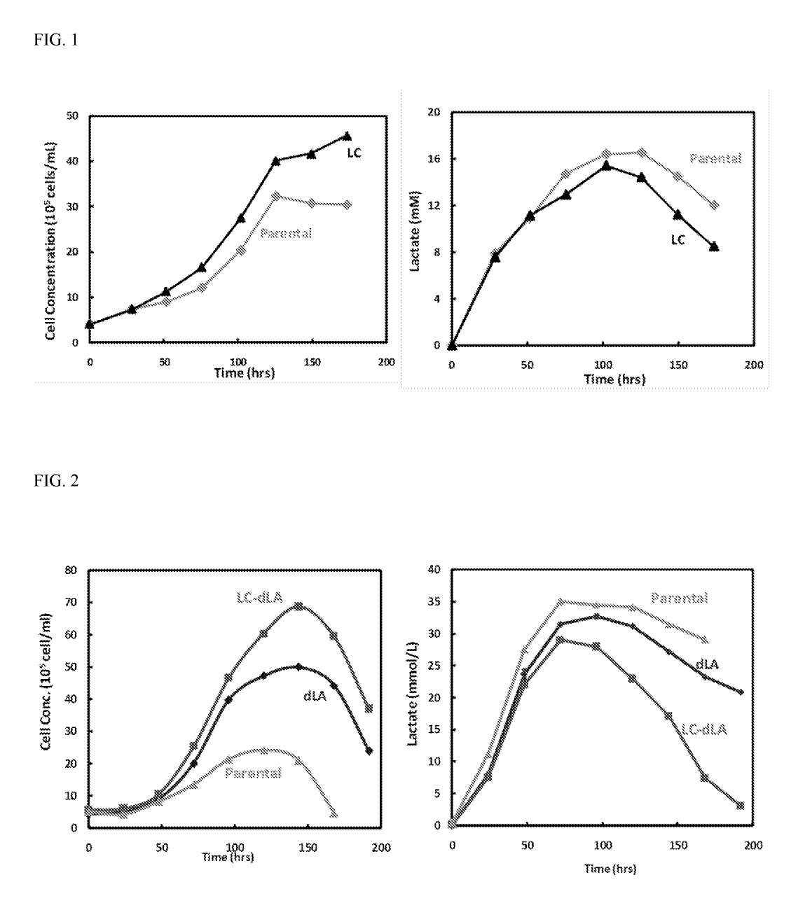

Next, we engineered a cell line that overexpressed the LDH-C transcript. The kinetic properties of this enzyme favor the conversion of lactate to pyruvate, and overexpression of LDH-C in the cell could potentially lead to lactate consumption, thereby reducing cellular concentrations of this inhibitory metabolite. As model systems, we used a parental antibody-producing SP2/0 cell line, as well as one of the newly-generated LDH-A knockdown clones. The effects of LDH-C overexpression, as well as combined LDH-C overexpression and LDHA knockdown on cellular growth and metabolism were characterized. (Example 2, Example 3, FIG. 1, FIG. 2).

Cells that overexpress LDH-C can possess the ability to shift their metabolism from lactate production to lactate consumption. Thus, cells overexpressing LDH-C can consume lactate in late-stage culture, when the cellular glycolytic activity is low and the extracellular lactate levels are high, compared to cells that are not engineered to overexpress LDH-C. Lactate is a byproduct of mammalian cellular metabolism that is released into extracellular medium, accumulations of which are known to inhibit growth and protein production. Consumption of lactate by cells therefore reduces the exposure time to the toxic effects of lactate. Thus, a shift in the cellular metabolism in late-stage culture away from lactate production and toward lactate consumption can promote sustained culture viability and, in some cases, higher maximum cell densities, each of which can prolong the length of the culture, yield higher titers, and/or permit greater accumulation of desired metabolic products.

In the analysis of hundreds of runs of manufacturing processes involving mammalian cell culture, the cultures that switched to lactate consumption in late-stage had substantially higher productivity than those that failed to consume lactate.

In one aspect, therefore, the invention provides a genetically modified cell that overexpresses LDH-C. In general, the cell may be any type of cell: mammalian, avian, amphibian, piscine, microbial, etc., including a transformed bacterium or a transformed yeast.

The genetically modified cell may overexpress LDH-C by any suitable mechanism. For example, the cell may be genetically modified by endonuclease modification of the LDH-C coding region or LDH-C regulatory region so that the LDH-C is expressed at a level greater than the native expression of LDH-C by the cell in the absence of the genetic modification. Endonuclease modifications may be made by any suitable endonuclease such as, for example, a zinc-finger nuclease (ZFN) or meganuclease.

Alternatively, LDH-C overexpression may be achieved by a genetically modifying the cell to include a heterologous polynucleotide sequence that comprises a coding region that encodes at least a portion of a heterologous lactate dehydrogenase C polypeptide. As used herein, a heterologous lactate dehydrogenase C polypeptide is a polypeptide synthesized by expression of a heterologous polynucleotide. As used herein, a heterologous polynucleotide is a polynucleotide molecule whose origin is outside the cell prior to being genetically modified. As such, a heterologous polynucleotide may be a polynucleotide molecule derived from another organism, whether or not that organism is a member of another species. In other embodiments, a heterologous polynucleotide may be a polynucleotide molecule that is a copy of a polynucleotide sequence from the organism from which the genetically modified cell is derived, but is synthesized outside of the genetically modified cell such as, for example, in vitro or in another cell, tissue, or organ of the organism prior to being introduced into, and thereby genetically modifying, the cell.

In some cases, the genetically modified cell may express the heterologous lactate dehydrogenase C polypeptide to a greater extent than a wild-type control—e.g., a comparable cell, without the genetic modification (e.g., endonuclease modification and/or heterologous polynucleotide sequence). In other cases, the genetically modified cell may produce lactate to a lesser degree than a wild-type control produces lactate. In other cases, the genetically modified cell consumes lactate to a greater degree than a wild-type control consumes lactate. In other cases, the genetically modified cell may possess a greater maximum cell density than the maximum cell density of a wild-type control. In other cases, the genetically modified cell may possess more than one of the foregoing characteristics including, for example, all four characteristics or any combination of two or any combination of three of the characteristics.

In some embodiments, the genetically modified cell may further include a polynucleotide molecule that inhibits expression of lactate dehydrogenase A. Such a polynucleotide can include an inhibitory RNA molecule such as, for example, a small interfering RNA (siRNA) molecule, a microRNA (miRNA), a small hairpin RNA (shRNA), or an antisense RNA. Alternatively, the inhibiting polynucleotide molecule may be a DNA that either inhibits translation of a target mRNA or encodes an RNA that inhibits translation of a target mRNA. Tools for designing a polynucleotide sequence that inhibits expression of a known genomic sequence are well known to those of ordinary skill in the art.

In some embodiments, the inhibiting polynucleotide molecule can encode a siRNA molecule. In some of these embodiments, a DNA encoding a siRNA molecule can include the nucleic acid sequence of SEQ ID NO:1 or SEQ ID NO:2.

In some embodiments, the inhibiting polynucleotide molecule can include a polynucleotide that encodes a polypeptide that can inhibit expression of a target mRNA. Such polypeptides include, for example, zinc-finger nuclease (ZFN) or a meganuclease.

The genetically modified cell can include a heterologous polynucleotide that includes a coding region as described below with respect to the isolated polynucleotide aspect of the invention. That is, any of the coding regions suitable for use in the isolated polynucleotides described below in detail are suitable for inclusion in a heterologous polynucleotide for use in a genetically modified cell.

Thus, in some embodiments, the genetically modified cell can include a heterologous polynucleotide that includes a coding region that includes, for example, a sufficient portion of SEQ ID NO:4, SEQ ID NO:6, SEQ ID NO:10, SEQ ID NO:13, SEQ ID NO:15, SEQ ID NO:17, SEQ ID NO:19, or SEQ ID NO:21 to encode a lactate dehydrogenase C polypeptide. In certain embodiments, the genetically modified cell can include a heterologous polynucleotide that includes a sufficient portion of the coding region of SEQ ID NO:10 to encodes a lactate dehydrogenase C polypeptide.

In other embodiments, the genetically modified cell can include a heterologous polynucleotide that includes a coding region that encodes the amino acid sequence of SEQ ID NO:5, SEQ ID NO:7, SEQ ID NO:8, SEQ ID NO:9, SEQ ID NO:11, SEQ ID NO:14, SEQ ID NO:16, SEQ ID NO:18, SEQ ID NO:20, SEQ ID NO:22, or any fragment or biologically active analog of any of the foregoing that possesses measurable lactate dehydrogenase C activity. In one embodiment, the genetically modified cell can include a heterologous polynucleotide that includes a coding region that encodes the amino acid sequence of SEQ ID NO:11.

In some embodiments, the genetically modified cell can further include a heterologous polynucleotide that encodes a commercially relevant product such as, for example, a therapeutic agent such as, for example, a recombinant protein, a recombinant subunit vaccine, a recombinant whole virus vaccine, and an anti-idiotype antibody. Exemplary therapeutic agents include, for example, recombinant monoclonal antibodies (mAbs) and recombinant therapeutic proteins such as tissue plasminogen activator (TPA), factor VIII, erythropoietin (EPO), etc. Other exemplary commercially relevant products include industrial compounds, bioremediation compounds, biofuel compounds, and commodity chemicals.

In another aspect, the present invention provides an isolated polynucleotide that includes a coding region that encodes at least a portion of a lactate dehydrogenase C polypeptide, operably linked to a promoter. In Example 2 and Example 3, we describe a polynucleotide that includes a coding region that encodes a mouse lactate dehydrogenase C coding region (GenBank accession: X04752.1 GI:52885, SEQ ID NO:10). The invention may be practiced, however, using a polynucleotide that encodes any suitable lactate dehydrogenase C polypeptide. Exemplary polynucleotides include, for example, the coding region of any one of SEQ ID NO:4, SEQ ID NO:6, SEQ ID NO:10, SEQ ID NO:13, SEQ ID NO:15, SEQ ID NO:17, SEQ ID NO:19, or SEQ ID NO:21. The polynucleotide can be from any suitable mammalian source such as, for example, mouse, human, rat, Chinese hamster, or Syrian hamster.

Other exemplary polynucleotides include those that encode a lactate dehydrogenase C polypeptide such as, for example, lactate dehydrogenase polypeptides that include the amino acid sequence of SEQ ID NO:5, SEQ ID NO:7, SEQ ID NO:8, SEQ ID NO:9, SEQ ID NO:11, SEQ ID NO:14, SEQ ID NO:16, SEQ ID NO:18, SEQ ID NO:20, SEQ ID NO:22, or any fragment or biologically active analog of any of the foregoing that possesses measurable lactate dehydrogenase C activity. In one embodiment, the polynucleotide encodes the amino acid sequence of SEQ ID NO:11.

Thus, a lactate dehydrogenase C polypeptide can include a native, full-length lactate dehydrogenase C protein or any fragment or biologically active subunit thereof that retains measurable LDH-C activity such as, for example, catalytic conversion of lactate +NAD⁺ to pyruvate +NADH. A “biologically active analog” of lactate dehydrogenase C can include one or more amino acid substitutions compared to a reference lactate dehydrogenase C amino acid sequence. Substitutes for an amino acid in the polypeptides of the invention may be selected from other members of the class to which the amino acid belongs. For example, it is well-known in the art of protein biochemistry that an amino acid belonging to a grouping of amino acids having a particular size or characteristic (such as charge, hydrophobicity and hydrophilicity) can be substituted for another amino acid without altering the activity of a protein, particularly in regions of the protein that are not directly associated with biological activity. Substitutes for an amino acid may be selected from other members of the class to which the amino acid belongs. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Examples of such preferred conservative substitutions include Lys for Arg and vice versa to maintain a positive charge; Glu for Asp and vice versa to maintain a negative charge; Ser for Thr so that a free—OH is maintained; and Gln for Asn to maintain a free NH2. A “biologically active analog” also includes a polypeptide having a deletion or addition of any number of amino acids, again so long as the analog retains measurable LDH-C activity. Moreover, LDH-C has been characterizes sufficiently to permit one to determine regions of the LDH-C amino acid sequence that may be deleted or otherwise altered and still retain measurable LDH-C activity. Likewise, the characterization of LDH-C alerts one to regions of the enzyme that are essential for function and are less tolerate of modification.

In another aspect, the present invention provides a method the generally increases the ability of a cell designed to produce a commercially relevant product—e.g., a therapeutic product—to produce that product. In this aspect, the invention provides a method that includes modifying a cell that produces a commercially relevant product. Generally, the method includes introducing into the cell an isolated polynucleotide that includes a coding region that encodes a lactate dehydrogenase C polypeptide. Generally, this method may be practiced using any type of cell designed to produce any type of commercially relevant product.

In some of these embodiments, the transformed cell expresses lactate dehydrogenase C polypeptide to a greater extent than the cell expressed lactate dehydrogenase C prior to being transformed. In other embodiments, the transformed cell consumes lactate to a greater degree than the cell consumed lactate before being transformed. In other embodiments, the transformed cell possesses a greater maximum cell density than the maximum cell density of the cell before being transformed. In other embodiments, the transformed cell may exhibit increased viability in culture. In still other embodiments, the genetically modified cell may possess more than one of the foregoing characteristics including, for example, all four characteristics or any combination of any two of the characteristics or any combination of any three of the characteristics. In embodiments, in which the genetically modified cell exhibits, for example, increased maximum cell density and/or increased viability, the method may increase the production of the commercially relevant product compared to the cell prior to being transformed. Thus, practicing this method can increase the microbial production and/or yield of, for example, therapeutic agents, industrial compounds, bioremediation compounds, biofuel compounds, and/or commodity chemicals.

In some embodiments, the transformed cells exhibit reduced lactate accumulation compared to the lactate accumulated a comparable control—i.e., the cells in culture prior to being transformed as described herein. In some embodiments, the reduced lactate accumulation can be no more than 75% of the lactate accumulated by a comparable control culture. Thus, cells grown as described herein can accumulate lactate at a level no more than 75%, no more than 70%, no more than 65%, no more than 60%, no more than 55%, no more than 50%, no more than 45%, no more than 40%, no more than 35%, no more than 30%, no more than 25%, no more than 20%, no more than 15%, no more than 10%, no more than 9%, no more than 8%, no more than 7%, no more than 6%, no more than 5%, no more than 4%, no more than 3%, no more than 2%, or no more than 1% of the lactate accumulated by a comparable control culture. In some cases, reduced lactate accumulation may be the result of the cells releasing less lactate into the culture medium. In other cases, reduced lactate accumulation may result from the cells consuming lactate from the culture medium. Whether bacterial cells consume lactate may be determined by routine methods. In some cases, reduced lactate accumulation may result from a combination of releasing less lactate into the culture medium and consuming lactate from the culture medium.

In some embodiments, the transformed cells can exhibit increased viability compared to a comparable control culture—i.e., the cells in culture prior to being transformed as described herein. In some embodiments, increased viability can be expressed in terms of the duration of the culture at which a particular percentage of the cells are measured as viable. Viable cells may be measured by any suitable, routine method including, for example, dye exclusion methods such as the method described in the Examples, below. In some embodiments, the percentage of viable cells that defines the “viability” of the culture can be at least 50% viable cells, at least 60% viable cells, at least 70% viable cells, at least 75% viable cells, at least 76% viable cells, at least 77% viable cells, at least 78% viable cells, at least 78% viable cells, at least 80% viable cells, at least 81% viable cells, at least 82% viable cells, at least 83% viable cells, at least 84% viable cells, or at least 85% viable cells. Thus, increased viability of the culture can be expressed as an increase in the duration that a culture of transformed cells maintains any desired level of “viability,” as defined immediately above, compared to a comparable control culture. In some cases, the viability may be increased by more than two-fold. Exemplary increases in the duration of maintaining viability of a culture can include, for example, increases of at least 10%, at least 20%, at least 30%, at least 40%, at least 50%, at least 60%, at least 70%, at least 80%, at least 90%, at least 100% (i.e., doubling), at least 125%, at least 150%, at least 170%, at least 200%, at least 225%, at least 250%, at least 275%, at least 300%, at least 500%, or at least 1000%.

Increased viability of the culture also can be expressed in terms of an increase in the percentage of viable cells at a given point in the culture. Thus, other exemplary increases in the viability of a culture can include an increase in the percentage of viable cells at, for example, 30 hours, 40 hours, 50 hours, 60 hours, 70 hours, 80 hours, 90 hours, 100, hours, 110 hours, 120 hours, 130 hours, 140 hours, 150 hours, 180 hours, 200 hours, 240 hours, 300 hours, or 360 hours of culture

In some embodiments, the transformed cells can exhibit a higher maximum cell density compared to a comparable control culture—i.e., the cells in culture prior to being transformed as described herein. Exemplary maximum increases in maximum cell density can be, for example, an increase of at least 10%, at least 20%, at least 30%, at least 40%, at least 50%, at least 60%, at least 70%, at least 80%, at least 90%, or at least 100% (i.e., doubling). Exemplary minimum increases in cell density also include an increase of no more than 200%, no more than 150%, no more than 100%, no more than 75%, no more than 50%, no more than 25%, or no more than 10%. In some embodiments, the increase in maximum cell density can fall within a range having endpoints defined by any maximum increase in maximum cell density provided above in combination with any appropriate minimum increase in maximum cell density provided above.

In some embodiments, transformed cells can exhibit greater accumulation of a recombinant product compared to a comparable control—e.g., compared to the accumulation of the product from cells lacking a transformation described herein. The increase in product accumulation can be, for example, a minimum increase of at least 2% compared to a comparable control. Thus, exemplary minimum increases in product accumulation include, for example, an increase of at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22, at least 23%, or at least 24%. In some embodiments, the increase in product accumulation may be expressed in terms of a fold increase over a comparable control or as a percentage increase over a comparable control. As used herein, for example, a doubling of the product accumulated can be expressed as a two-fold increase or as a 100% increase in the accumulation of the product. Accordingly, the increase in product accumulation may be a maximum increase of no more than a 100-fold increase compared to a comparable control. Thus, exemplary increases in product accumulation can be, for example, an increase of no more than 50-fold, no more than 20-fold, no more than ten-fold, no more than five-fold, or no more than two-fold, no more than 50%, no more than 25%, no more than 20%, no more than 19%, no more than 18%, no more than 17%, no more than 16%, no more than 15%, no more than 14%, no more than 13%, no more than 12%, no more than 11%, no more than 10%, no more than 9%, no more than 8%, no more than 7%, no more than 6%, no more than 5%, no more than 4%, no more than 3%, or no more than 2% over a comparable control. In some embodiments, the increase in product accumulation can fall within a range having endpoints defined by any maximum increase in product accumulation provided above in combination with any appropriate minimum increase in product accumulation provided above.

In yet another aspect, the present invention provides an alternative method for controlling lactate accumulation in batch culture. In this aspect, the method generally involves growing cells in batch culture while controlling the amount of glucose provided to the culture medium. In particular, the method involves controlling the amount of glucose in the culture to a low glucose concentration, described in detail below, beginning generally at a point during the late exponential phase or at a point thereafter. This method may be used for growing any suitable type of cell, including any embodiment of the genetically modified cells described above.

Controlling the glucose concentration can induce the cells to shift to a metabolic state that is characterized by reduced lactate accumulation and/or enhanced lactate consumption compared to growth of the cells in a comparable culture medium that includes glucose at a concentration of at least 2.0 g/L or uncontrolled levels of glucose (for brevity hereafter, collectively referred to as “high” glucose concentrations). Such a metabolic shift can allow cells to grow to higher maximum cell densities and/or maintain viability for longer periods than when the cells are provided glucose in an uncontrolled manner or in a controlled manner but at higher concentrations of glucose. In embodiments in which the cells are genetically modified to produce a product of interest, achieving a higher cell density and/or extending cell viability can result in a greater accumulation of the product.

The concentration of glucose in the culture may be controlled beginning from the late exponential phase of fedbatch cultures or at any time thereafter. As used herein, “late exponential phase” refers to the point at which the growth rate of the culture falls from its maximum growth rate to a growth rate that is no more than 20% of the maximum growth rate. As used herein, “growth rate” refers to the ratio of bacterial growth to bacterial death in a culture—i.e., the net growth rate. The precise timing of late exponential phase can vary depending upon many factors including, for example, the organism being cultured, the medium, the initial growth rate of the culture, and the culture conditions (e.g., temperature, atmosphere, shaking, volume, lactic acid concentration, etc.). However, one can approximate the late exponential phase of a batch culture by monitoring the cell density of the culture such as, for example, by monitoring the optical density of a sample from the culture. Such methods are routine for those of ordinary skill in the art. Thus, in some embodiments, the method can include determining the growth rate of the culture at one or more time points. However, with experience, one may be able to approximate the maximum growth rate and/or the point at which the growth rate falls to a point at which one desires to initiate control of the glucose concentration.

Control of the glucose concentration may be initiated after the growth rate of the culture falls from the maximum growth rate to a growth rate of no more than 20% of the maximum growth rate. Thus, in some embodiments, control of the glucose concentration may be initiated when the growth rate of the culture falls from the maximum to a growth rate of 20% of the maximum growth rate. In other embodiments, however, control of the glucose concentration may be initiated at any time after the growth rate has slowed to 20% of the maximum growth rate. Thus, in some embodiments, control of the glucose concentration may be initiated when the growth rate of the culture falls to 15%, 10%, 9%, 8%, 7%, 6%, 5%, 4%, 3%, 2%, or 1% of the maximum growth rate of the culture. In other embodiments, control of the glucose concentration may be initiated when the culture enters stationary phase, a phase defined as no net growth of the culture—i.e., when bacterial growth equals bacterial death.

As used herein, “low glucose concentration” refers to a culture medium containing measurable glucose at a non-zero concentration of no more than 1.0 g/L such as, for example, no more than 0.9 g/L, no more than 0.8 g/L, no more than 0.7 g/L, no more than 0.6 g/L, no more than 0.5 g/L, no more than 0.4 g/L, no more than 0.3 g/L, no more than 0.2 g/L, no more than 0.1 g/L, no more than 0.09 g/L, no more than 0.08 g/L, no more than 0.07 g/L, no more than 0.06 g/L, no more than 0.05 g/L, no more than 0.04 g/L, no more than 0.03 g/L, no more than 0.02 g/L, or no more than 0.01 g/L. In some embodiments, the glucose concentration can have a maximum of no more than 0.5 g/L such as, for example, no more than 0.4 g/L, no more than 0.3 g/L, no more than 0.2 g/L, no more than 0.1 g/L, no more than 0.09 g/L, no more than 0.08 g/L, or no more than 0.07 g/L. In some embodiments, the glucose concentration can have a minimum of at least 0.01 g/L such as, for example, at least 0.2 g/L, at least 0.3 g/L, at least 0.4 g/L, at least 0.5 g/L, at least 0.6 g/L, at least 0.7 g/L, at least 0.8 g/L, at least 0.9 g/L, or at least 1.0 g/L. In some embodiments, the glucose concentration can fall within a range having endpoints defined by any maximum glucose concentration provided above in combination with any appropriate minimum glucose concentration provided above.

In some embodiments, controlling the glucose concentration as described above can result in a culture in which the cells exhibit reduced lactate accumulation at a given time point compared to a viable culture of the same cells grown in comparable culture medium that contains higher concentrations of glucose—i.e. uncontrolled levels of glucose or controlled glucose concentrations of at least 2.0 g/L. In some embodiments, the reduced lactate accumulation can be no more than 75% of the lactate accumulated by a comparable viable control culture—i.e., the same cells grown at higher glucose concentrations. Thus, cells grown as described herein can accumulate lactate at a level no more than 75%, no more than 70%, no more than 65%, no more than 60%, no more than 55%, no more than 50%, no more than 45%, no more than 40%, no more than 35%, no more than 30%, no more than 25%, no more than 20%, no more than 15%, no more than 10%, no more than 9%, no more than 8%, no more than 7%, no more than 6%, no more than 5%, no more than 4%, no more than 3%, no more than 2%, or no more than 1% of the lactate accumulated by a comparable control culture. In some cases, reduced lactate accumulation may be the result of the cells releasing less lactate into the culture medium. In other cases, reduced lactate accumulation may result from the cells consuming lactate from the culture medium. Whether bacterial cells consume lactate may be determined by routine methods. In some cases, reduced lactate accumulation may result from a combination of releasing less lactate into the culture medium and consuming lactate from the culture medium.

In some embodiments, controlling the glucose concentration as described above can result in a culture that exhibits increased viability compared to a culture of the same cells cultured in comparable medium under high glucose conditions. For example, referring to FIG. 3, the low glucose culture maintained at least 50% viable cells for 300 hours, whereas the uncontrolled glucose culture maintained 50% viable cells for less than 200 hours. This effect is even more pronounced when one considers the duration of viability from the point at which control of the glucose concentration was initiated (approximately 120 hours). As used herein, the point at which control of the glucose concentration is initiated refers to the point in a culture when one begins controlling the glucose concentration in a low glucose culture or the comparable point in a comparable uncontrolled glucose or otherwise high glucose culture. Thus, the term refers to a point in time where treatment of controlled glucose cultures and high glucose cultures diverge and does not necessarily require the control of the glucose concentration in any particular—e.g., high glucose—culture. Considered from the point at which control of the glucose concentration is initiated, the low glucose culture maintained at least 50% viable cells for 180 hours after initiation of glucose concentration control, while the uncontrolled glucose culture maintained 50% viable cells for approximately 80 hours after the same time point.

One also can consider the culture viability in terms of the percentage of viable cells at a given time in the culture. FIG. 3 shows that the percentage of viable cells in the low glucose culture begins to diverge from the percentage of viable cells in the uncontrolled glucose culture at approximately 150 hours, some 30 hours after the control of the glucose concentration was initiated in the low glucose culture. The divergence was maintained throughout the remaining life of the culture.

Thus, in some embodiments, controlling the glucose concentration results in a culture in which at least 50% of the cells remain viable for longer than the time that 50% of the cells remain viable in a comparable high glucose culture. Thus, in some embodiments, the percentage of viable cells that defines the “viability” of the culture can be at least 50% viable cells, at least 60% viable cells, at least 70% viable cells, at least 75% viable cells, at least 76% viable cells, at least 77% viable cells, at least 78% viable cells, at least 78% viable cells, at least 80% viable cells, at least 81% viable cells, at least 82% viable cells, at least 83% viable cells, at least 84% viable cells, or at least 85% viable cells.

Increased viability of the culture can be expressed as an increase in the duration that a controlled low glucose culture maintains any desired level of “viability” as defined immediately above. In some cases, the viability may be increased by more than two-fold—e.g., the increase in maintaining 50% viable cells from approximately 80 hours to 180 hours after initiating control of the glucose concentration. Exemplary increases in the duration of maintaining viability of a culture can include, for example, increases of at least 10%, at least 20%, at least 30%, at least 40%, at least 50%, at least 60%, at least 70%, at least 80%, at least 90%, at least 100% (i.e., doubling), at least 125%, at least 150%, at least 170%, at least 200%, at least 225%, at least 250%, at least 275%, at least 300%, at least 500%, or at least 1000%.

Increased viability of the culture also can be expressed in terms of an increase in the percentage of viable cells at a given time after the time at which control of the glucose concentration is initiated, as defined herein. Thus, other exemplary increases in the viability of a culture can include an increase in the percentage of viable cells at, for example, 30 hours, 40 hours, 50 hours, 60 hours, 70 hours, 80 hours, 90 hours, 100, hours, 110 hours, or 120 hours after control of the glucose concentration is initiated.

In some embodiments, controlling the glucose concentration as described above can result in a culture that exhibits a higher maximum cell density compared to a comparable control culture—i.e., the same cells grown at a high glucose concentration. Exemplary maximum increases in maximum cell density can be, for example, an increase of at least 10%, at least 20%, at least 30%, at least 40%, at least 50%, at least 60%, at least 70%, at least 80%, at least 90%, or at least 100% (i.e., doubling). Exemplary minimum increases in cell density also include an increase of no more than 200%, no more than 150%, no more than 100%, no more than 75%, no more than 50%, no more than 25%, or no more than 10%. In some embodiments, the increase in maximum cell density can fall within a range having endpoints defined by any maximum increase in maximum cell density provided above in combination with any appropriate minimum increase in maximum cell density provided above.

In some embodiments, controlling the glucose concentration as described above can result in a culture that exhibits greater accumulation of a recombinant product compared to a comparable control—e.g., compared to the accumulation of the product when the cells are grown in a comparable culture medium at higher glucose levels. The increase in product accumulation can be, for example, a minimum increase of at least 2% compared to a comparable control. Thus, exemplary minimum increases in product accumulation include, for example, an increase of at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22, at least 23%, or at least 24%. In some embodiments, the increase in product accumulation may be expressed in terms of a fold increase over a comparable control or as a percentage increase over a comparable control. As used herein, for example, a doubling of the product accumulated can be expressed as a two-fold increase or as a 100% increase in the accumulation of the product. Accordingly, the increase in product accumulation may be a maximum increase of no more than a 100-fold increase compared to a comparable control. Thus, exemplary increases in product accumulation can be, for example, an increase of no more than 50-fold, no more than 20-fold, no more than ten-fold, no more than five-fold, or no more than two-fold, no more than 50%, no more than 25%, no more than 20%, no more than 19%, no more than 18%, no more than 17%, no more than 16%, no more than 15%, no more than 14%, no more than 13%, no more than 12%, no more than 11%, no more than 10%, no more than 9%, no more than 8%, no more than 7%, no more than 6%, no more than 5%, no more than 4%, no more than 3%, or no more than 2% over a comparable control. In some embodiments, the increase in product accumulation can fall within a range having endpoints defined by any maximum increase in product accumulation provided above in combination with any appropriate minimum increase in product accumulation provided above.

The method may be applied to any cell culture processes used to produce a commercially relevant product such as, for example, a therapeutic protein or vaccine. Further, the method can be extended to processes in which the cells are the end product of the culture. For example, the production of stem cells for cellular therapy may benefit from the redirection of cellular metabolism to produce less lactate.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### General Methods

### Microarray Data

Total RNA from two Chinese hamster ovary cell lines of different parental origin (DG44 and DXB11) was harvested from mid-exponential stage samples using the RNeasy Mini kit (Qiagen Inc. Valencia, Calif.). Ovaries from late adolescent (4-month-old) virgin female Chinese hamsters were used for total RNA isolation using Trizol (Invitrogen, Carlsbad, Calif.).

RNA was isolated using RNeasy mini kits (Qiagen, Valencia, Calif.) according to the manufacturer's protocol. Biotin-labeled cRNA was prepared from 5 μg of total RNA from each sample using the one-cycle target labeling (Affymetrix, Inc., Santa Clara, Calif.) according to the manufacturer's instructions. Labeled, fragmented cRNA was hybridized on CHO Affymetrix arrays (version 1 with 10,118 probe sets), washed, and scanned at the University of Minnesota Affymetrix Microarray Core Facility. CEL files were processed using the GeneData Expressionist Refiner module (GeneData AG, San Francisco, Calif.), which was used to assess overall array quality and obtain a single intensity values for each probe set using the Microarray Analysis Suite Statistical Algorithm (MAS 5.0). The mean intensity values for all chips were linearly scaled to 500. Genes with a maximum intensity ≦70 and a detection p-value ≧0.04 across all samples in a given study were called absent and excluded from further analysis. Probe sets with a detection p-value <0.04 and intensity >70 in at least one sample were retained for analysis.

Total RNA from biological replicate cultures of mid-exponential growing SP2/0 cells expressing a recombinant IgG was extracted using the RNeasy Mini kit (Qiagen Inc., Valencia, Calif.) and labeled using the Affymetrix One Sample labeling kit as described above. Samples were hybridized onto Affymetrix MOE 430 2.0 arrays containing 45,023 probe sets. Intensity data was extracted as described above. Finally, microarray data from mouse plasma cells were downloaded from NCBI's Gene Expression Omnibus microarray data repository (GDS1695), and quadruplet samples hybridized onto MOE 420 2.0 arrays were used for analysis. Data were scaled to an average intensity of 500 to be comparable across samples.

Fold changes between cultured cell lines and tissue samples were calculated for each system (CHO and SP2/0) and visualized in the context of metabolic and signaling pathways using GenMAPP (Dahlquist et al., 2002).

### Cell Culture

The SP2/0 cell line produces a recombinant antibody product and has been described previously (Sauer et al., 2000). The parental SP2/0 cells and transfected clones were maintained in T-flasks and grown in a medium based on DMEM/F12 (1:1), containing glucose (21.7 mM), glutamine (6.25 mM), sodium bicarbonate (29 mM), putrescine (0.7 μM), penicillin G (0.17 mM), streptomycin (68.6 μM), pluronic F68 (60 μM) and phenol red (19.9 μM).

### Batch and Fed-Batch Cultures

Exponentially growing cells from T-flask seed cultures were inoculated at a concentration of 4×105 cells/mL. Samples were taken daily for the duration of the cultures. For each sample, cell concentration and viability were determined by counting with a hematocytometer using trypan blue staining Lactate concentrations were measured using the YSI Model 27 industrial analyzer (YSI Inc., Yellow Springs, Ohio). Glucose concentrations were determined in duplicate using Infinity Glucose Hexokinase Reagent (Thermo Electron Corp., Waltham, Mass.) according to the manufacturer's protocol. Absorbance was read at 340 nm using a SpectraMax Plus 384 plate reader (Molecular Devices, Inc., Sunnyvale, Calif.).

Antibody IgG concentrations in cell culture supernatants were measured by ELISA. Goat anti-human IgG Fc-specific and mouse anti-goat IgG Alkaline phosphatase (Sigma-Aldrich, St. Louis, Mo.) were used as primary and secondary detection antibodies, while P-nitro-phenyl phosphate was used as the enzyme substrate (Sigma-Aldrich, St. Louis, Mo.) and human IgG (Sigma-Aldrich, St. Louis, Mo.) was used as a standard. IgG concentration was determined by absorbance reading at 405 nm.

For fed-batch cultures, 1 mL of feed medium was added from Day 3 onwards. The feed medium composition was ten-fold (10×) concentrated basal medium excluding bulk salts (NaHCO₃, NaCl, CaCl₂, KCl).

Calculations of cumulative and specific consumption and based on mass balance equations for all nutrients, metabolites and cells present in the culture. Cumulative consumption or production of nutrients and metabolites were calculated as follows:

$${S}_{i , t} = {\int }_{0}^{t} {q}_{i , t} \cdot x \cdot V \cdot d t \approx {V}_{{t}_{0}} \cdot {S}_{i , {t}_{0}} - {V}_{t} \cdot {S}_{i , t} + {\sum }_{0}^{k} {V}_{fk} \cdot {S}_{fk}$$

where:
S_{i,t}: cumulative amount of nutrient i consumed or produced at time t,
V: culture volume,
Sᵢ: concentration of component i in the culture medium,
V_{f}: volume of feed medium added,
S_{f}: concentration of component i in the feed medium, and
k: the total number of feed medium additions up until time t.

Specific consumption or production rates of nutrients and metabolites were calculated as follows:

$${q}_{s} = \frac{1}{x \cdot V} \frac{d S}{d t}$$

A third-order polynomial function was fitted to each component's cumulative consumption data and the fitted equations were used to take the derivative and calculate the specific rate. Specific consumption and production rates were determined by plotting the substrate or product concentrations against the time integral values of the growth curve and calculating the slope (Renard et al., 1988).

For batch cultures grown in low glucose concentrations, cells were seeded at 3.5×10⁵ cells/mL in 6-well plates, and parallel cultures were initiated in growth medium containing either 0.1 g/L or 4 g/L glucose concentrations. Cells were cultured for 12 hours, after which all samples were counted, and glucose and lactate concentrations were measured, as described above.

### Culture Assays

Cell densities were determined by direct cell counting using a hemacytometer. Cell viability was determined using Trypan blue exclusion (Invitrogen, Carlsbad, Calif.). Lactate was measured using a YSI 2700 Biochemistry Analyzer (YSI, Inc., Yellow Springs, Ohio). Antibody Was measured by ELISA as follows: Goat anti-human IgG Fc-specific and mouse anti-goat IgG Alkaline phosphatase (Sigma-Aldrich, St. Louis, Mo.) were used as primary and secondary detection antibodies, while P-nitro-phenyl phosphate was used as the enzyme substrate (Sigma-Aldrich, St. Louis, Mo.) and human IgG (Sigma-Aldrich, St. Louis, Mo.) was used as a standard. IgG concentration was determined by absorbance reading at 405 nm.

### Example 1

### Vector Construction, Transfection and Clone Isolation for LDH-A Knockdown

The Invitrogen Block-iT™ Pol II miR RNAi expression system (K4935-00) was used for knockdown. This system allows the expression of knockdown cassettes driven by RNA polymerase II (Pol II) promoters. miRNA sequences designed against the gene of interest are flanked by native miRNA sequences which allow for proper processing of the miRNA transcript. The Invitrogen RNAi designer software (https://rnaidesigner.invitrogen.com/rnaiexpress/) was used to design targeting sequences against the mouse LDH-A gene (NM_{—}010699) and mouse LDH-B gene (NM_{—}008492).

The top two sequences against LDH-A and the top sequence against LDH-B, as ranked by the software, were used for targeting. The LDH-A targeting sequences were CAAGGACCAGCTGATTGTGAA (SEQ ID NO:1, named LDH-A1) and ACGTGAACATCTTCAAGTTCA (SEQ ID NO:2, named LDH-A2), while the LDH-B targeting sequence was AGTCTCCCTCCAGGAACTGAA (SEQ ID NO:3, named LDH-B). Each targeting sequence was inserted into the targeting vector, pcDNA6.2-GW/EmGFP-miR (Invitrogen, Carlsbad, Calif.). Briefly, 200 μM of top strand oligo and 200 μM of bottom strand oligo were combined with annealing buffer to a total volume of 20 μL and incubated at 95° C. for four minutes. This mixture was diluted 5.000-fold and used for ligation: 10 ng of linearized vector was combined with 10 nM double-stranded oligos, ligation buffer and 1 U T4 DNA ligase and the reaction was allowed to proceed for 5 minutes at room temperature. 2 μL of the ligation mixture was combined with one vial of ONESHOT TOP10 chemically competent E. coli cells (Invitrogen, Carlsbad, Calif.), incubated on ice for five minutes and heat-shocked for 30 seconds at 42° C. 250 μL of room temperature super optimal broth (SOB) medium (Invitrogen, Carlsbad, Calif.) was added to the cells and incubated for one hour at 37° C. with shaking 20 μL of bacterial culture was plated onto pre-warmed LB agar plates containing 50 μg/mL spectinomycin and incubated overnight at 37° C.

Ten colonies for each expression construct were expanded, and plasmid DNA was purified using the QIAGEN Plasmid Mini Kit (Qiagen inc. Valencia, Calif.). Constructs were sequenced to verify the presence and correct orientation of the insert, as well as the sequence of the insert using the forward sequencing primer provided with the kit. Upon sequence confirmation, one E. coli clone for each sequencing construct was expanded and frozen glycerol stocks were established. Large quantities of plasmid DNA were obtained using 1 L bacterial cultures and purified using the QIAGEN Plasmid Maxi kit (Qiagen inc. Valencia, Calif.). A negative control vector was also constructed in parallel using the same procedure as described above, and named pcDNA6.2-Neg. The negative control targeting sequence, which represents a scrambled sequence, is provided with the kit as a top and bottom strand oligo.

The RNAi expression system allows for up to three targeting sequences to be chained together on the same plasmid. Consequently, the three targeting sequences (LDH-A1, LDH-A2, LDH-B) were combined into one expression vector. Briefly, 2 μg of pcDNA6.2-LDH-A2 was digested with 10 U BamH I and 10 U Xho I for 2 hour at 37° C. In parallel, 2 μg of pcDNA6.2-LDH-A1 was digested with 10 U Bgl II and 10 U Xho I for 2 hour at 37° C. Digests were run on 2% agarose gels, and the backbone and insert fragments were excised from the gel and purified using the QIAquick Gel Extraction kit (Qiagen Inc., Valencia, Calif.). The purified backbone and insert were ligated at a 1:4 molar ratio using 10 U T4 DNA ligase. ONESHOT TOP10 chemically competent E. coli (Invitrogen, Carlsbad, Calif.) cells were transformed as described above. Resultant colonies were expanded and plasmid DNA was purified using the QIAGEN Plasmid Mini Kit (Qiagen Inc. Valencia, Calif.). Constructs were sequenced and verified. One E. coli clone was selected, expanded, and a frozen glycerol stock was established. The resulting construct was named pcDNA6.2-LDH-A1A2. To further append the LDHB targeting construct, the same procedure as described above was used, using pcDNA6.2-LDH-B as the insert and pcDNA6.2-LDH-A1A2 as the backbone. The resulting construct was sequence verified and named pcDNA6.2-LDH-A1A2B.

40 μg of plasmid (pcDNA6.2-Neg, pcDNA6.2-LDH-A1, pcDNA6.2-LDH-A2, pcDNA6.2-LDH-B, pcDNA6.2-LDH-A1A2 or pcDNA6.2-LDH-A1A2B) was linearized by digestion with 10 U of Pci I, and purified using QIAQuick PCR purification spin columns (Qiagen, Valencia, Calif.). Ten million exponentially growing cells were washed twice in 10 mL of cold Opti-MEM medium (Invitrogen, Carlsbad, Calif.), and electroporated with 40 μg of linearized DNA in 1 mL of cold Opti-MEM in 4 mm electroporation cuvettes (BioRad Laboratories, Inc., Hurcules, Calif.). Electroporation was performed in the Gene Pulser XceII (BioRad Laboratories, Inc., Hurcules, Calif.). The electroporation conditions used for SP2/0 cells were 300 V, 250 μF and infinite resistance. Transfected cells were transferred into pre-warmed growth medium supplemented with 10% FBS (Atlas Biological, Inc. Ft. Collins, Colo.). Transfection efficiency was determined using FACS by parallel transfection with an EGFP-containing plasmid.

Transfected cells were diluted in 96-well plates at 2000 cells/well, in 0.2 mL of maintenance medium supplemented with 4 μg/mL blasticidin. Plates were incubated for 10-12 days in a 37° C., 5% CO2 environment. Clones were expanded for characterization in their selective medium.

### Confirming Knockdown

RNA was isolated using RNEasy columns (Qiagen, Inc., Valencia, Calif.) according to the manufacturer's protocol, with on-column DNAse digestion (Qiagen™, Inc., Valencia, Calif.). cDNA synthesis was performed from 5 μg of total RNA using Superscript III Reverse transcriptase (Invitrogen, Carlsbad, Calif.). Primers for the mouse 18s rRNA (control), LHD-A and LDH-B genes were designed using Primer3 (Steve Rozen and Helen J. Skaletsky (2000) Primer3 on the WWW for general users and for biologist programmers. In: Krawetz S, Misener S (eds) Bioinformatics Methods and Protocols: Methods in Molecular Biology. Humana Press, Totowa, N.J., pp 365-386) with a specified product size of 150 to 250 bp and melting temperature of 60° C. Quantitative real-time PCR was performed using the STRATAGENE Mx3000P (Agilent Technologies, Inc., Santa Clara, Calif.) with SYBR Green I dye chemistry using the STRATAGENE Full Velocity SYBR green QPCR kit (Agilent Technologies, Inc., Santa Clara, Calif.). PCR conditions were: 94° C. for 10 minutes, followed by 40 cycles of 95° C. for 30 seconds, 60° C. for one minute, and 72° C. for 30 seconds.

Dissociation curves were determined after PCR by complete dissociation at 95° C., one minute, followed and 30 seconds annealing at 55° C. and a rapid temperature ramp to 95° C. The Ct (threshold cycle number) values were determined at 0.2 of the reference dye normalized baseline value. Triplicate cDNA samples, a no RT-reaction control, and a no cDNA template control were run for each sample/primer pair. All PCR products were run on 2% agarose gels to confirm expected product sizes.

To confirm knockdown at the enzyme activity level, LDH activity was measured using a colorimetric LDH assay (Sigma-Aldrich, St. Louis, Mo., catalog #TOX7). This assay is based on the reduction of NAD+ by LDH, and subsequent stoichiometric conversion of a tetrazolium dye by NADH. Briefly, 8×10⁴ cells from each sample were isolated and resuspended in 100 μL growth medium. 0.1× volume of LDH assay lysis solution was added to each sample and incubated at 37° C. for 45 minutes. Cells were spun to pellet debris, and supernatant was transferred to 96-well plate. LDH assay mixture was prepared by combining equal volumes of LDH assay substrate, cofactor prep and dye solution. 2× volume of this mixture was added to each sample and incubated at room temperature, in the dark for 30 minutes. The reaction was terminated by adding 0.1× volume of 1 N HCL. Absorbance was measured at 490 nm. A series of dilution was performed for each sample and a straight line was fit to each dilution vs. absorbance curve. Percent knockdown was determined based on percent change of the slope of the straight line fit.

### Example 2

### Vector Construction, Transfection and Clone Isolation for LDH-C Overexpression

The full-length isoform C of the mouse LDH gene (GenBank accession: X04752.1, SEQ ID NO:10) was obtained in the pDNR-Lib vector from Open Biosystems (Open Biosystems Products, Hunstville, Ala.). The coding sequence was subcloned into the pcDNA_{—}3.1_bsd vector (Invitrogen, Carlsbad, Calif.) through restriction digest with EcoR I and Xho I to yield pcDNA_{—}3.1_bsd_LDHC. The resulting construct was transfected into two cell lines in parallel: the parental SP2/0 cell line, as well as the LDH-A knockdown clone B4 (described above). A control transfection was also carried out in these two cells by transfecting the empty vector, pcDNA_{—}3.1_bsd. Transfection and selection conditions used in this study were the same as those described above.

### Confirming LDH-C Overexpression

Quantitative real-time PCR was used to confirm overexpression of the target gene in the isolated clones. Primers for the mouse LHD-C gene were designed using Primer3 (Steve Rozen and Helen J. Skaletsky (2000) Primer3 on the WWW for general users and for biologist programmers. In: Krawetz S, Misener S (eds) Bioinformatics Methods and Protocols: Methods in Molecular Biology. Humana Press, Totowa, N.J., pp 365-386) with a specified product size of 150 to 250 bp and melting temperature of 60° C. PCR conditions were the same as described above.

### Example 3

Recombinant antibody producing mouse myeloma SP2/0 cell line was genetically modified as described in Example 1 for LDH-C over-expression. FIG. 1 shows the growth characteristic and lactate profiles from fed-batch cultures of LDH-C overexpression (LC) cell line. Fed-batch cultures were performed using 125 mL shaker flasks which were daily fed with concentrated medium, starting Day 3. In comparison to parental clone, by the end of 100 hours, LC cell line reached higher cell concentration and exhibited lower lactate levels. Beyond 100 hours, LC cell line demonstrated higher lactate consumption than the parental cell line.

FIG. 2 shows growth and lactate profiles of cell line LC-dLA which over expresses LDH-C on a LDH-A knockdown background. The over-expression of LDH C was confirmed by quantitative PCR using LDH-C specific primer. The transcript level of LDH-C is 4350-fold higher in LC-dLA line than the parental line, which does not have either LDH A knockdown or LDH C over-expression. In fed-batch cultures using 125 mL shaker flasks daily feeding with concentrated medium (3% volume) was started on Day 2. Cell concentration and lactate concentration profiles for LC-dLA and dLA, as well as parental lines are shown. All the three cell lines grew exponentially reaching maximum cell concentrations around Day 5 and Day 6. LC-dLA line reached a higher maximum cell concentration than either dLA or parental cell line.

During their exponential growth phase all the three lines produced lactate, which accumulated to somewhat different levels. LC-dLA has the lowest lactate accumulation among the three lines. After the peak growth was reached, a metabolic shift to lactate consumption was observed as indicated by the decreasing concentration of lactate. Lactate consumption was greatest in the LC-dLA culture.

### Example 4

CHO cells are transfected using plasmid-containing mouse LDH-C gene using the neomycin resistance as selectable marker. The surviving cells are assessed for the genome integration of the trans-gene (LDH-C) and the mRNA transcript levels of LDH-C. Fed-batch cultures are performed to evaluate the ability of LDH-C expressing CHO cells to shift their metabolism in the late-stage of the culture towards lactate consumption. CHO cells overexpressing LDH-C will exhibit greater late-stage cell density, greater lactate consumption, and lower lactate accumulation than untransfected CHO cells.

### Example 5

Batch cultures were used to grow either CHO cell line that expresses a model IgG or a mouse myeloma cell line (SP2/0) that expressed a model IgG. DMEM:F12 (1:1) basal medium was used for maintaining, expanding, and inoculating cells in the bioreactor. The pH of the culture was maintained at 7.0 using carbon-dioxide gas injection or addition of 1 N NAOH solution. The temperature was maintained at 37° C. by heating coil wrapped around bioreactor. Dissolved oxygen (DO) was maintained at 30% of air saturation by adjusting the oxygen concentration in the inlet gas. Overall oxygen transfer coefficient (KLa) was determined for the reactor stepup and was used for estimating cellular oxygen uptake rate (OUR).

Bioreactors were fed a fixed volume of a chemically-defined feed medium (about 10-fold concentrated as compared to basal media) starting on Day 2 until Day 5. On Day 5 the culture was split into two, one was continuously fed with concentrated media at low rates, so as to maintain culture glucose concentration at less than 0.04 g/L. The other culture was maintained at a high glucose level by adding a bolus of concentrated media every day. The bioreactor was sampled just after inoculation and every day throughout the run. The following off-line measurements were made from each fresh 5-mL sample: glucose, lactate, viable cell density, cell viability, and antibody titer. Results for the CHO cell line are shown in FIG. 3.

During the exponential growth phase until Day 5, lactate was produced continuously. With the onset of controlling low glucose level, a metabolic shift to lactate consumption was observed, which continued through the end of the bioreactor culture. In contrast, cells in high glucose conditions did not shift to lactate consumption metabolic state and viability dropped below 50% by Day 8. Unlike the high glucose culture, the metabolic shift to lactate consumption in low glucose culture extended the viability of the culture and sustained higher cell concentrations through Day 14. As an outcome, low glucose culture accumulated 20% higher titer than the high glucose culture (166 mg of total protein as compared to 133 mg of protein in case of high glucose culture). Results are sown in FIG. 3.

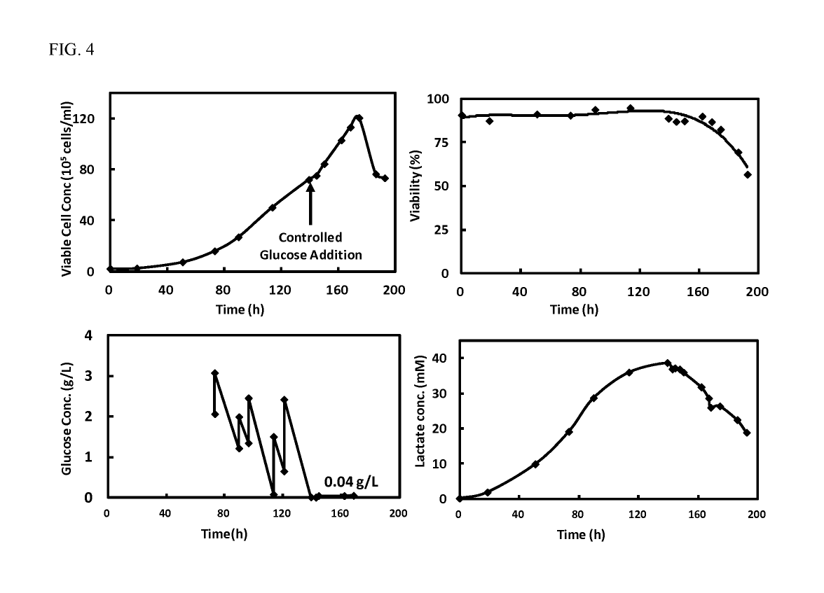

Fedbatch culture of mouse myeloma (SP2/0) cell line producing IgG was carried out. Glucose concentration was maintained at low levels starting from Day 5 bp controlled addition of concentrated feed media. Cells grew exponentially, reaching maximum cell concentrations of approximately 8×10⁶ cells/ml on Day 5. During their exponential growth phase lactate was produced continuously. With the onset of the controlled, low glucose conditions, a metabolic shift to lactate consumption was observed, which was observed through the end of the bioreactor culture. Significant amount of lactate was consumed by the end of the bioreactor run. Results are shown in FIG. 4.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. An isolated polynucleotide comprising:
a coding region that encodes at least a portion of a lactate dehydrogenase C polypeptide, operably linked to a promoter.

2. The isolated polynucleotide of claim 1 wherein the coding region comprises at least a portion of SEQ ID NO:4, SEQ ID NO:6, SEQ ID NO:10, SEQ ID NO:13, SEQ ID NO:15, SEQ ID NO:17, SEQ ID NO:19, or SEQ ID NO:21 that encodes at least a portion of a lactate dehydrogenase C polypeptide.

3. The isolated polynucleotide of claim 2 wherein the coding region comprises at least a portion of SEQ ID NO:10 that encodes at least a portion of a lactate dehydrogenase C polypeptide.

4. The isolated polynucleotide of claim 1 wherein the coding region encodes the amino acid sequence of SEQ ID NO:5, SEQ ID NO:7, SEQ ID NO:8, SEQ ID NO:9, SEQ ID NO:11, SEQ ID NO:14, SEQ ID NO:16, SEQ ID NO:18, SEQ ID NO:20, or SEQ ID NO:22.

5. The isolated polynucleotide of claim 4 wherein the coding region encodes the amino acid sequence of SEQ ID NO:11.

6. A genetically modified cell that comprises greater lactate dehydrogenase C activity compared to a wild-type control.

7. A genetically modified cell that consumes lactate to a greater degree than a wild-type control.

8. A genetically modified cell that produces lactate to a lesser degree than a wild-type control.

9. A genetically modified cell that comprises a greater maximum cell density when grown in nutrient-rich medium compared to a wild-type control.

10. The genetically modified cell of claim 6 wherein the cell comprises a heterologous polynucleotide molecule that comprises an isolated polynucleotide comprising a coding region that encodes at least a portion of a lactate dehydrogenase C polypeptide, operably linked to a promoter.

11. The genetically modified cell of claim 6 wherein the cell further exhibits reduced lactate dehydrogenase A activity compared to a wild-type control.

12. The genetically modified cell of claim 10 further comprising an inhibiting polynucleotide molecule that inhibits expression of lactate dehydrogenase A.

13. The genetically modified cell of claim 12 wherein the inhibiting polynucleotide molecule comprises SEQ ID NO:1 or SEQ ID NO:2.

14. The genetically modified cell of claim 12 wherein the inhibiting polynucleotide molecule encodes a polypeptide that inhibits expression of lactate dehydrogenase A.

15. The genetically modified cell of claim 10 wherein the heterologous polynucleotide comprises a coding region that comprises at least a portion of SEQ ID NO:4, SEQ ID NO:6, SEQ ID NO:10, SEQ ID NO:13, SEQ ID NO:15, SEQ ID NO:17, SEQ ID NO:19, or SEQ ID NO:21 that encodes at least a portion of a lactate dehydrogenase C polypeptide.

16. The genetically modified cell of claim 15 wherein the coding region comprises at least a portion of SEQ ID NO:10 that encodes at least a portion of a lactate dehydrogenase C polypeptide.

17. The genetically modified cell of claim 10 wherein the heterologous polynucleotide comprises a coding region that encodes the amino acid sequence of SEQ ID NO:5, SEQ ID NO:7, SEQ ID NO:8, SEQ ID NO:9, SEQ ID NO:11, SEQ ID NO:14, SEQ ID NO:16, SEQ ID NO:18, SEQ ID NO:20, or SEQ ID NO:22.

18. The genetically modified cell of claim 17 wherein the coding region encodes the amino acid sequence of SEQ ID NO:11.

19. The genetically modified cell of claim 6 further comprising a heterologous polynucleotide sequence that encodes a therapeutic agent.

20. The genetically modified cell of claim 6 comprising:
a lactate dehydrogenase C coding region; and
an endonuclease modification that results in the lactate dehydrogenase C coding region being expressed to a greater degree than a comparable cell without the endonuclease modification.

21. A method comprising:
providing a cell that produces a therapeutic product; and
introducing into the cell the isolated polynucleotide of claim 1, thereby producing a transformed cell.

22. The method of claim 21 wherein introducing the isolated polynucleotide into the cell causes the cell to increase expression of lactate dehydrogenase C.

23. The method of claim 21 wherein introducing the isolated polynucleotide into the cell causes the cell to decrease production of lactate.

24. The method of claim 21 wherein introducing the isolated polynucleotide into the cell causes the cell to increase consumption of lactate.

25. The method of claim 21 wherein introducing the isolated polynucleotide into the cell causes an increase in the cell's maximum cell density.

26. A method comprising:
growing a cell in culture comprising culture medium comprising glucose; and
controlling glucose concentration in the culture medium so that the culture exhibits at least one of the following characteristics compared to growth of the cell in culture medium comprising at least 2.0 g/L glucose: reduced lactate accumulation, enhanced lactate consumption, increase cell density, or increased viability.

27. The method of claim 26 wherein:
growing the cell in culture comprises determining the growth rate of the culture at one or more time points; and
initiating controlling the glucose concentration at a time when or after the growth rate of the culture falls from a maximum growth rate to 20% of the maximum growth rate.

28. The method of claim 26 wherein the cell comprises a genetically modified cell that comprises greater lactate dehydrogenase C activity compared to a wild-type control.

29. The method of claim 26 wherein the glucose concentration in the culture is no more than 0.5 g/L.


SEQUENCE LISTING (22 sequences)

SEQ ID NO: 1 - DNA, 21 bp, Artificial Sequence
  DNA encoding an siRNA molecule inhibiting lactate dehydrogenase A
caaggaccagctgattgtgaa

SEQ ID NO: 2 - DNA, 21 bp, Artificial Sequence
  DNA encoding an siRNA molecule inhibiting lactate dehydrogenase A
acgtgaacatcttcaagttca

SEQ ID NO: 3 - DNA, 21 bp, Artificial Sequence
  DNA encoding an siRNA molecule inhibiting lactate dehydrogenase B
agtctccctccaggaactgaa

SEQ ID NO: 4 - DNA, 1318 bp, Homo sapiens
cgtgcgtgtctcgagtcgcacggagggcaaccgtcgacgggcttagcgcctcaactgtcgttggtgtatttttctggtgtcacttctgtgccttccttcaaaggtggtgctttgtccctgtgggtcatctgtactgattgcgccaagcaaagcatttgttctccaaatgtcaactgtcaaggagcagctaattgagaagctaattgaggatgatgaaaactcccagtgtaaaattactattgttggaactggtgccgtaggcatggcttgtgctattagtatcttactgaaggatttggctgatgaacttgcccttgttgatgttgcattggacaaactgaagggagaaatgatggatcttcagcatggcagtcttttctttagtacttcaaagattacttctggaaaagattacagtgtatctgcaaactccagaatagttattgtcacagcaggtgcaaggcagcaggagggagaaactcgccttgccctggtccaacgtaatgtggctataatgaaatcaatcattcctgccatagtccattatagtcctgattgtaaaattcttgttgtttcaaatccagtggatattttgacatatatagtctggaagataagtggcttacctgtaactcgtgtaattggaagtggttgtaatctagactctgcccgtttccgttacctaattggagaaaagttgggtgtccaccccacaagctgccatggttggattattggagaacatggtgattctagtgtgcccttatggagtggggtgaatgttgctggtgttgctctgaagactctggaccctaaattaggaacggattcagataaggaacactggaaaaatatccataaacaagttattcaaagtgcctatgaaattatcaagctgaaggggtatacctcttgggctattggactgtctgtgatggatctggtaggatccattttgaaaaatcttaggagagtgcacccagtttccaccatggttaagggattatatggaataaaagaagaactctttctcagtatcccttgtgtcttggggcggaatggtgtctcagatgttgtgaaaattaacttgaattctgaggaggaggcccttttcaagaagagtgcagaaacactttggaatattcaaaaggatctaatattttaaattaaagccttctaatgttccactgtttggagaacagaagatagcaggctgtgtattttaaattttgaaagtattttcatttgatctttaaaaaataaaaacaaattggagacctgtgaaaaaaaaaaaaaaaaaaaaaaaaaaaaaaaaaaa

SEQ ID NO: 5 - protein, 330 aa, Homo sapiens
MSTVKEQLIEKLIEDDENSQCKITIVGTGAVGMACAISILLKDLADELALVDVALDKLKGEMMDLQHGSLFFSTSKITSGKDYSVSANSRIVIVTAGARQQEGETRLALVQRNVAIMKSIIPAIVHYSPDCKILWSNPVDILTYIVWKISGLPVTRVIGSGCNLDSARFRYLIGEKLGVHPTSCHGWIIGEHGDSSVPLWSGVNVAGVALKTLDPKLGTDSDKEHWKNIHKQVIQSAYEIIKLKGYTSWAIGLSVMDLVGSILKNLRRVHPVSTMVKGLYGIKEELFLSIPCVLGRNGVSDWKINLNSEEEALFKKSAETLWNIQKDLFF

SEQ ID NO: 6 - DNA, 1264 bp, Homo sapiens
cgtgcgtgtctcgagtcgcacggagggcaaccgtcgacgggcttagcgcctcaactgtcgttggtgtatttttctggtgtcacttctgtgccttccttcaaaggttctccaaatgtcaactgtcaaggagcagctaattgagaagctaattgaggatgatgaaaactcccagtgtaaaattactattgttggaactggtgccgtaggcatggcttgtgctattagtatcttactgaaggatttggctgatgaacttgcccttgttgatgttgcattggacaaactgaagggagaaatgatggatcttcagcatggcagtcttttctttagtacttcaaagattacttctggaaaagattacagtgtatctgcaaactccagaatagttattgtcacagcaggtgcaaggcagcaggagggagaaactcgccttgccctggtccaacgtaatgtggctataatgaaatcaatcattcctgccatagtccattatagtcctgattgtaaaattcttgttgtttcaaatccagtggatattttgacatatatagtctggaagataagtggcttacctgtaactcgtgtaattggaagtggttgtaatctagactctgcccgtttccgttacctaattggagaaaagttgggtgtccaccccacaagctgccatggttggattattggagaacatggtgattctagtgtgcccttatggagtggggtgaatgttgctggtgttgctctgaagactctggaccctaaattaggaacggattcagataaggaacactggaaaaatatccataaacaagttattcaaagtgcctatgaaattatcaagctgaaggggtatacctcttgggctattggactgtctgtgatggatctggtaggatccattttgaaaaatcttaggagagtgcacccagtttccaccatggttaagggattatatggaataaaagaagaactctttctcagtatcccttgtgtcttggggcggaatggtgtctcagatgttgtgaaaattaacttgaattctgaggaggaggcccttttcaagaagagtgcagaaacactttggaatattcaaaaggatctaatattttaaattaaagccttctaatgttccactgtttggagaacagaagatagcaggctgtgtattttaaattttgaaagtattttcatttgatctttaaaaaataaaaacaaattggagacctgtgaaaaaaaaaaaaaaaaaaaaaaaaaaaaaaaaaaa

SEQ ID NO: 7 - protein, 330 aa, Homo sapiens
MSTVKEQLIEKLIEDDENSQCKITIVGTGAVGMACAISILLKDLADELALVDVALDKLKGEMMDLQHGSLFFSTSKITSGKDYSVSANSRIVIVTAGARQQEGETRLALVQRNVAIMKSIIPAIVHYSPDCKILWSNPVDILTYIVWKISGLPVTRVIGSGCNLDSARFRYLIGEKLGVHPTSCHGWIIGEHGDSSVPLWSGVNVAGVALKTLDPKLGTDSDKEHWKNIHKQVIQSAYEIIKLKGYTSWAIGLSVMDLVGSILKNLRRVHPVSTMVKGLYGIKEELFLSIPCVLGRNGVSDWKINLNSEEEALFKKSAETLWNIQKDLIF

SEQ ID NO: 8 - protein, 330 aa, Mus musculus
MSTVKEQLIQNLVPEDKLSRCKITWGVGNVGMACAISILLKGLADELALVDADTNKLRGEALDLLHGSLFLSTPKIVFGKDYNVSANSKLVIITAGARMVSGETRLDLLQRNVAIMKAIVPGIVQNSPDCKIIIVTNPVDILTYWWKISGFPVGRVIGSGCNLDSARFRYLIGEKLGVNPTSCHGWVLGEHGDSSVPIWSGVNVAGVTLKSLNPAIGTDSDKEHWKNVHKQVVEGGYEVLNMKGYTSWAIGLSVTDLARSILKNLKRVHPVTTLVKGFHGIKEEVFLSIPCVLGQSGITDFVKVNMTAEEEGLLKKSADTLWNMQKDLQL

SEQ ID NO: 9 - protein, 332 aa, Mus musculus
MSTVKEQLIQNLVPEDKLSRCKITVVGVGNVGMACAISILLKGLADELALVDADTNKLRGEALDLVHGSLFLSTPKIVFGKDYNVSANSKLVIITAGARMVSGETRLDLLQRNVAIMKAIVPGIVQNSPDCKIIIVTNPVDILTYVVWKISGFPVGRVIGSGCNLDSARFRYLIGEKLGVNPTSCHGWVLGEHGDSSVPIWSGVNVAGVTLKSLNPAIGTDSDKEHWKNVHKQVVEGGYEVLNMKGYTSWAIGLSVTDLARSILKNLKRVHPVTTLVKGFHGIKEEVFLSIPCVLGQSGITDFVKVNMTAEEEGLLKKSADTLWNMQKDLQL

SEQ ID NO: 10 - DNA, 1167 bp, Mus musculus
caacagttatcctggtttcttacctgtgctgcggagtcagcagtaaggctcaacatgtccaccgtcaaggagcagctgattcagaacctagttccggaagataaactttcccggtgtaagattactgtggtcggagttggaaatgtgggcatggcgtgtgctattagtattttactgaagggtttggctgatgaacttgcccttgttgacgctgatacgaacaaactgaggggagaggcactggatcttctgcacggcagtcttttccttagcactccaaaaatcgtctttggaaaagattacaatgtatctgccaactccaaactggttattatcacagctggtgcaagaatggtgtctggagaaactcgccttgacctgctccaacgtaatgtcgctatcatgaaagccattgttccgggcattgtccaaaacagtccggactgtaaaataattatcgtcactaacccagtggatattttgacatacgtggtttggaagataagcggcttccctgtaggccgtgtgatcggaagtggctgtaacctagactcagcacgttttcgttacctgattggggagaagctgggtgtcaaccctacaagctgccacggctgggttcttggagaacatggggactccagtgtgcccatatggagtggtgtaaacgttgctggcgtaactctgaagtcactgaacccagcaataggaactgactcagataaggaacactggaaaaatgttcacaagcaggtggtggaaggcggctatgaggtccttaacatgaagggctatacctcttgggctatcgggctgtctgtgactgatctggcgcgatccatcttgaagaatcttaagagagtgcatcctgttaccacgctggttaagggcttccatgggataaaggaagaggtcttcctcagtatcccttgtgtcttgggacaaagtggtatcacagactttgtgaaagtcaacatgaccgctgaggaggagggtctcctcaagaagagtgcggacacactctggaatatgcagaaggatctgcagttataaactcgccaccttcgaccgtgtgacagatgcctgatcacatcactgatcacggcagtcccactgaaagtgtttccacatcataacaaagttcaataaaattttggaaacctgtta

SEQ ID NO: 11 - protein, 332 aa, Mus musculus
MSTVKEQLIQNLVPEDKLSRCKITVVGVGNVGMACAISILLKGLADELALVDADTNKLRGEALDLLHGSLFLSTPKIVFGKDYNVSANSKLVIITAGARMVSGETRLDLLQRNVAIMKAIVPGIVQNSPDCKIIIVTNPVDILTYVVWKISGFPVGRVIGSGCNLDSARFRYLIGEKLGVNPTSCHGWVLGEHGDSSVPIWSGVNVAGVTLKSLNPAIGTDSDKEHWKNVHKQVVEGGYEVLNMKGYTSWAIGLSVTDLARSILKNLKRVHPVTTLVKGFHGIKEEVFLSIPCVLGQSGITDFVKVNMTAEEEGLLKKSADTLWNMQKDLQL

SEQ ID NO: 12 - protein, 328 aa, Mus musculus
MSTVKEQLIQNLVPEDKLSRCKITWGVGNVGMACAISILLKGLADELALVDADTNKLRGEALDLLHGSLFLSTPKIVFGKDYNVSANSKLVIITAGARMVSGETRLDLLQRNVAIMKAIVPGIVQNSPDCKIIIVTNPVDILTYWWKISGFPVGRVIGSGCNLDSARFRYLIGEKLGVNPTSCHGWVLGEHGDSSVPIWSGVNVAGVTLKSLNPAIGTDSDKEHWKNVHKQWEGGYEVLNMKGYTSWAIGLSVTDLARSILKNLKRVHPVTTLVKGFHGIPEEVFLSIPCVLGQSGITDFVKVNMTAEEEGLLKKSADTLWNMQKDLQ

SEQ ID NO: 13 - DNA, 1133 bp, Mus musculus
agcagaaggctcaacatgtccaccgtcaaggagcagctgattcagaacctagttccggaagataaactttcccggtgtaagattactgtggtcggagttggaaatgtgggcatggcgtgtgcaattagtattttactgaagggtttggctgatgaacttgcccttgttgacgctgatacgaacaaactgaggggagaggcactggatcttctgcacggcagtcttttccttagcactccaaaaatcgtctttggaaaagattacaatgtatctgccaactccaaactggttattatcacagctggtgcaagaatggtgtctggagaaactcgccttgacctgctccaacgtaatgtcgctatcatgaaagccattgttccgggcattgtccaaaacagtccggactgtaaaataattatcgtcactaacccagtggatattttgacatacgtggtttggaagataagcggcttccctgtaggccgtgtgatcggaagtggctgtaacctagactcagcacgttttcgttacctgattggggagaagctgggtgtcaaccctacaagctgccacggctgggttcttggagaacatggggactccagtgtgcccatatggagtggtgtaaacgttgctggcgtaactctgaagtcactgaacccagcaataggaactgactcagataaggaacactggaaaaatgttcacaagcaggtggtggaaggcggctatgaggtccttaacatgaagggctatacctcttgggctatcgggctgtctgtgactgatctggcgcgatccatcttgaagaatcttaagagagtgcatcctgttaccacgctggttaagggcttccatgggataaaggaagaggtcttcctcagtatcccttgtgtcttgggacaaagtggtatcacagactttgtgaaagtcaacatgaccgctgaggaggagggtctcctcaagaagagtgcggacacactctggaatatgcagaaggatctgcagttataaactcgccaccttcgaccgtgtgacagatgcctgatcacatcactgatcacggcggtcccactgaaagtgtttccacatcataacaaagttcaataaaattttggaaacctgttaagatc

SEQ ID NO: 14 - protein, 329 aa, Mus musculus
MSTVKEQLIQNLVPEDKLSRCKITWGVGNVGMACAISILLKGLADELALVDADTNKLRGEALDLLHGSLFLSTPKIVFGKDYNVSANSKLVIITAGARMVSGETRLDLLQRNVAIMKAIVPGIVQNSPDCKIIIVTNPVDILTYWWKISGFPVGRVIGSGCNLDSARFRYLIGEKLGVNPTSCHGWVLGEHGDSSVPIWSGVNVAGVTLKSLNPAIGTDSDKEHWKNVHKQWEGGYEVLNMKGYTSWAIGLSVTDLARSILKNLKRVHPVTTLVKGFHGIKEEVFLSIPCVLGQSGITDFVKVNMTAEEEGLLKKSADTLWNMQKDLQL

SEQ ID NO: 15 - DNA, 1140 bp, Mus musculus
agcagaaggctcaacatgtccaccgtcaaggagcagctgattcagaacctagttccggaagataaactttcccggtgtaagattactgtggtcggagttggaaatgtgggcatggcgtgtgcaattagtattttactgaagggtttggctgatgaacttgcccttgttgacgctgatacgaacaaactgaggggagaggcactggatcttctgcacggcagtcttttccttagcactccaaaaatcgtctttggaaaagattacaatgtatctgccaactccaaactggttattatcacagctggtgcaagaatggtgtctggagaaactcgccttgacctgctccaacgtaatgtcgctatcatgaaagccattgttccgggcattgtccaaaacagtccggactgtaaaataattatcgtcactaacccagtggatattttgacatacgtggtttggaagataagcggcttccctgtaggccgtgtcatcggaagtggctgtaacctagactcagcacgttttcgttacctgattggggagaagctgggtgtcaaccctacaagctgccacggctgggttcttggagaacatggggactccagtgtgcccatatggagtggtgtaaacgttgctggcgtaactctgaagtcactgaacccagcaataggaactgactcagataaggaacactggaaaaatgttcacaagcaggtggtggaaggcggctatgaggtccttaacatgaagggctatacctcttgggctatcgggctgtctgtgactgatctggcgcgatccatcttgaagaatcttaagagagtgcatcctgttaccacgctggttaagggcttccatgggataccggaagaggtcttcctcagtatcccttgtgtcttgggacaaagtggtatcacagactttgtgaaagtcaacatgaccgctgaggaggagggtctcctcaagaagagtgcggacacactctggaatatgcagaaggatctgcagttataaactcgccaccttcgaccgtgtgacagatgcctgatcacatcactgatcacggcggtcccactgaaagtgtttccacatcataacaaagttcaataaaattttggaaacctgttaagatcaaaaaaa

SEQ ID NO: 16 - protein, 331 aa, Mus musculus
MSTVKEQLIQNLVPEDKLSRCKITWGVGNVGMACAISILLKGLADELALVDADTNKLRGEALDLLHGSLFLSTPKIVFGKDYNVSANSKLVIITAGARMVSGETRLDLLQRNVAIMKAIVPGIVQNSPDCKIIIVTNPVDILTYVVWKISGFPVGRVIGSGCNLDSARFRYLIGEKLGVNPTSCHGWVLGEHGDSSVPIWSGVNVAGVTLKSLNPAIGTDSDKEHWKNVHKQVVEGGYEVLNMKGYTSWAIGLSVTDLARSILKNLKRVHPVTTLVKGFHGIPEEVFLSIPCVLGQSGITDFVKVNMTAEEEGLLKKSADTLWNMQKDLQL

SEQ ID NO: 17 - DNA, 1199 bp, Mus musculus
agttatcctggtttcttacctgtgctgcggagtcagcagtaaggctcaacatgtccaccgtcaaggagcagctgattcagaacctagttccggaagataaactttcccggtgtaagattactgtggtcggagttggaaatgtgggcatggcgtgtgctattagtattttactgaagggtttggctgatgaacttgcccttgttgacgctgatacgaacaaactgaggggagaggcactggatcttctgcacggcagtcttttccttagcactccaaaaatcgtctttggaaaagattacaatgtatctgccaactccaaactggttattatcacagctggtgcaagaatggtgtctggagaaactcgccttgacctgctccaacgtaatgtcgctatcatgaaagccattgttccgggcattgtccaaaacagtccggactgtaaaataattatcgtcactaacccagtggatattttgacatacgtggtttggaagataagcggcttccctgtaggccgtgtgatcggaagtggctgtaacctagactcagcacgttttcgttacctgattggggagaagctgggtgtcaaccctacaagctgccacggctgggttcttggagaacatggggactccagtgtgcccatatggagtggtgtaaacgttgctggcgtaactctgaagtcactgaacccagcaataggaactgactcagataaggaacactggaaaaatgttcacaagcaggtggtggaaggcggctatgaggtccttaacatgaagggctatacctcttgggctatcgggctgtctgtgactgatctggcgcgatccatcttgaagaatcttaagagagtgcatcctgttaccacgctggttaagggcttccatgggataaaggaagaggtcttcctcagtatcccttgtgtcttgggacaaagtggtatcacagactttgtgaaagtcaacatgaccgctgaggaggagggtctcctcaagaagagtgcggacacactctggaatatgcagaaggatctgcagttataaactcgccaccttcgaccgtgtgacagatgcctgatcacatcactgatcacggcagtcccactgaaagtgtttccacatcataacaaagttcaataaaattttggaaacctgttaagatcgaaaaaaaaaaaaaaaaaaaaaaaaaaaaaa

SEQ ID NO: 18 - protein, 329 aa, Mus musculus; identical to SEQ ID NO: 14

SEQ ID NO: 19 - DNA, 1152 bp, Mus musculus
gacctgtgctgcggagtcagcagtaaggctcaacatgtccaccgtcaaggagcagctgattcagaacctagttccggaagataaactttcccggtgtaagattactgtggtcggagttggaaatgtgggcatggcgtgtgctattagtattttactgaagggtttggctgatgaacttgcccttgttgacgctgatacgaacaaactgaggggagaggcactggatcttctgcacggcagtcttttccttagcactccaaaaatcgtctttggaaaagattacaatgtatctgccaactccaaactggttattatcacagctggtgcaagaatggtgtctggagaaactcgccttgacctgctccaacgtaatgtcgctatcatgaaagccattgttccgggcattgtccaaaacagtccggactgtaaaataattatcgtcactaacccagtggatattttgacatacgtggtttggaagataagcggcttccctgtaggccgtgtgatcggaagtggctgtaacctagactcagcacgttttcgttacctgattggggagaagctgggtgtcaaccctacaagctgccacggctgggttcttggagaacatggggactccagtgtgcccatatggagtggtgtaaacgttgctggcgtaactctgaagtcactgaacccagcaataggaactgactcagataaggaacactggaaaaatgttcacaagcaggtggtggaaggcggctatgaggtccttaacatgaagggctatacctcttgggctatcgggctgtctgtgactgatctggcgcgatccatcttgaagaatcttaagagagtgcatcctgttaccacgctggttaagggcttccatgggataaaggaagaggtcttcctcagtatcccttgtgtcttgggacaaagtggtatcacagactttgtgaaagtcaacatgaccgctgaggaggagggtctcctcaagaagagtgcggacacactctggaatatgcagaaggatctgcagttataaactcgccaccttcgaccgtgtgacagatgcctgatcacatcactgatcacggcagtcccactgaaagtgtttccacatcataacaaagttcaataaaattttggaaacctgttaagacc

SEQ ID NO: 20 - protein, 329 aa, Mus musculus; identical to SEQ ID NO: 14

SEQ ID NO: 21 - DNA, 1311 bp, Mus musculus
gatcctggtttcttacctgtgctgcggagtcagcagtaaggctcaacatgtccaccgtcaaggagcagctgattcagaacctagttccggaagataaactttcacggtgtaagattactgtggtcggagttggaaatgtgggcatggcgtgtgctattagtattttactgaagggtttggctgatgaacttgcccttgttgacgctgatacgaacaaactgaggggagaggcactggatcttctgcacggcagtcttttccttagcactccaaaaatcgtctttggaaaagattacaatgtatctgccaactccaaactggttattatcacagctggtgcaagaatggtgtctggagaaactcgccttgacctgctccaacgtaatgtcgctatcatgaaagccattgttccgggcattgtccaaaacagtccggactgtaaaataattatcgtcactaacccagtggatattttgacatacgtggtttggaagataagcggcttccctgtaggccgtgtgatcggaagtggctgtaacctagactcagcacgttttcgttacctgattggggagaagctgggtgtcaaccctacaagctgccacggctgggttcttggagaacatggggactccagtgtgcccatatggagtggtgtaaacgttgctggcgtaactctgaagtcactgaacccagcaataggaactgactcagataaggaacactggaaaaatgttcacaagcaggtggtggaaggcggctatgaggtccttaacatgaagggctatacctcttgggctatcgggctgtctgtgactgatctggcgcgatccatcttgaagaatcttaagagagtgcatcctgttaccacgctggttaagggcttccatgggataaaggaagaggtcttcctcagtatcccttgtgtcttgggacaaagtggtatcacagactttgtgaaagtcaacatgaccgctgaggaggagggtctcctcaagaagagtgcggacacactctggaatatgcagaaggatctgcagttataaactcgccaccttcgaccgtgtgacagatgcctgatcacatcactgatcacggcagtcccactgaaagtgtttccacatcataacaaagttcaataaaattttggaaacctgttaagatcaatctcaaggctagaaagattaatgccaaaggcatctccctccccctttttttgagacagggtctcactttatagccttagctgacctcaaacggaaatctgctagcctcccagtgtattaaaggcaaccaccaccaggcccagct

SEQ ID NO: 22 - protein, 329 aa, Mus musculus; identical to SEQ ID NO: 14